(19) United States
(12) Patent Application Publication
(10) Pub. No.: US 2022/0015657 A1
(43) Pub. Date: Jan. 20, 2022

(54) PROCESSING EEG DATA WITH TWIN NEURAL NETWORKS

(71) Applicant: X Development LLC, Mountain View, CA (US)

(72) Inventors: Mustafa Ispir, Mountain View, CA (US); Pramod Gupta, Mountain View, CA (US); Garrett Raymond Honke, Mountain View, CA (US)

(21) Appl. No.: 16/933,219

(22) Filed: Jul. 20, 2020

(51) Int. Cl. A61B 5/04; A61B 5/00; G06N 3/04; G06N 3/08

(52) U.S. Cl. CPC A61B 5/04004; A61B 5/7275; G06N 3/049; G06N 3/0454; G06N 3/084; A61B 5/7264

(57) ABSTRACT

Methods, systems, and apparatus, including computer programs encoded on computer storage media, for generating embeddings of EEG measurements. One of the methods includes obtaining a plurality of electroencephalogram (EEG) signal measurements of a user, wherein each EEG signal measurement corresponds to one of a plurality of prompt types of an EEG task; generating, from the plurality of EEG signal measurements, a plurality of network inputs each corresponding to a different prompt type of the plurality of prompt types of the EEG task; processing the network inputs using a twin neural network to generate respective network outputs each corresponding to a different prompt type of the plurality of prompt types of the EEG task; and providing the network outputs to a downstream neural network to generate a mental health prediction for the user.

### BACKGROUND

This specification relates to generating outputs using neural networks.

Neural networks, or for brevity, networks, are machine learning models that employ multiple layers of operations to predict one or more outputs from one or more inputs. Neural networks typically include one or more hidden layers situated between an input layer and an output layer. The output of each layer is used as input to another layer in the network, e.g., the next hidden layer or the output layer.

Each layer of a neural network specifies one or more transformation operations to be performed on input to the layer. Some neural network layers have operations that are referred to as neurons. Each neuron receives one or more inputs and generates an output that is received by another neural network layer. Often, each neuron receives inputs from other neurons, and each neuron provides an output to one or more other neurons.

An architecture of a neural network specifies what layers are included in the network and their properties, as well as how the neurons of each layer of the network are connected. In other words, the architecture specifies which layers provide their output as input to which other layers and how the output is provided.

The transformation operations of each layer are performed by computers having installed software modules that implement the transformation operations. Thus, a layer being described as performing operations means that the computers implementing the transformation operations of the layer perform the operations.

Each layer generates one or more outputs using the current values of a set of parameters for the layer. Training the neural network thus involves continually performing a forward pass on the input, computing gradient values, and updating the current values for the set of parameters for each layer using the computed gradient values. Once a neural network is trained, the final set of parameter values can be used to make predictions in a production system.

### SUMMARY

This specification describes a system that processes electroencephalogram (EEG) signal measurements using a twin neural network to generate an embedding of the EEG signal measurements. In particular, the system can process EEG signal measurements corresponding to each of multiple prompt types of an EEG task, and generate a respective embedding corresponding to each prompt type of the EEG task. The embeddings of the EEG signal measurements can be provided to one or more downstream models for generating a mental health prediction of a user.

The subject matter described in this specification can be implemented in particular embodiments so as to realize one or more of the following advantages. Some implementations compute the difference between EEG signal measurements corresponding to different prompt types of an EEG task, and generate mental health predictions using the difference. Computing a raw difference between two EEG signal measurements might not capture all of the information encoded in the EEG signal measurements. Using techniques described in this disclosure, a system can train a twin neural network to automatically learn to extract meaningful information from EEG signal measurements to generate embeddings, and can train a downstream neural network to automatically determine a dissimilarity between the embeddings corresponding to different prompt types of an EEG task. Using a twin neural network allows a system to learn EEG-specific transformations corresponding to multiple different EEG tasks and EEG prompt types simultaneously, which allows the system to train the neural network using less training data.

By processing the EEG signal measurements corresponding to different prompt types using a twin neural network with matching parameter values, the generated embeddings can be directly compared to make mental health predictions for a user. For example, generating the respective embeddings corresponding to each prompt type of an EEG task using a twin neural network that has one-dimensional convolutional neural network layers can allow a system to more accurately generate a mental health prediction of a user. In some implementations, described in this specification, the same twin neural network can be used to generate respective embeddings of EEG data corresponding to multiple different EEG tasks.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a diagram of an example EEG embedding system.

FIG. 2 is a diagram of an example twin neural network for generating EEG embeddings.

FIGS. 3a and 3B are diagrams of example hierarchical twin neural networks for generating EEG embeddings corresponding to multiple EEG tasks.

FIG. 4 is a flow diagram of an example process for generating a mental health prediction for a user.

Like reference numbers and designations in the various drawings indicate like elements.

### DETAILED DESCRIPTION

This specification describes a system that processes EEG signal measurements using a twin neural network to generate an embedding of the EEG signal measurements. The embedding of the EEG signal measurements can be provided to one or more downstream models for generating a mental health prediction of a user.

In this specification, a mental health prediction of a user can be a prediction regarding any aspect of the mental health status of the user. For example, a mental health prediction can represent the likelihood that the user has one or more particular mental illnesses. As another example, a mental health prediction can represent a classification of a personality type of the user, e.g., a prediction that the user belongs to a particular one out of N possible personality types. As another example, the mental health prediction can represent a likelihood that the user will develop one or more illnesses or traits in the future, e.g., a likelihood that the user will develop a particular addiction. As another example, the mental health prediction can represent a prediction of a current mental state of the user, e.g., a prediction regarding attention allocation, anticipation, surprise, etc.

An EEG signal measurement characterizes brain activity of a user. Generally, multiple electrodes are placed on the scalp of the user at different locations, and each electrode measures electrical activity at the scalp over a period of time. This measured electrical activity represents the electrical activity of the brain at the corresponding location, and thus provides a measure of certain aspects of brain activity and function. The measurement captured by each electrode is a one-dimensional time series, where each element of the time series represents the level of electrical activity of the brain at the corresponding location at a particular time point. The time series captured by each electrode can be included in a respective channel of the EEG signal measurement. That is, a given EEG signal measurement characterizes the brain activity of the user during a particular period of time and includes one or more channels that each corresponds to a respective location on the scalp of the user.

In some cases, EEG signal measurements are captured while the user is performing a mental task, referred to in this specification as an “EEG task.” A system can present a prompt corresponding to the EEG task to the user, e.g., using a graphical user interface, and capture an EEG signal measurement in response to the prompt; the process of capturing an

EEG signal measurement is referred to in this specification as an “EEG trial.” In some cases, the EEG task can be passive, e.g., the prompt can be to look at an image. In some other cases, the EEG task can be active, e.g., the prompt can be to select a choice from a set of options.

An EEG task can have one or more different prompt types that represent different categories of prompts of the EEG task. Generally, the brain activity of the user in response to prompts of different categories will be different. As a particular example, an EEG task can be to view an image, a first prompt type can be to view pleasant images, and a second prompt type can be to view unpleasant images. As another example, an EEG task can be to view monetary rewards and losses, a first prompt type can be to view monetary rewards, and a second prompt type can be to view monetary losses.

An EEG signal measurement captured in response to a prompt of a first prompt type and an EEG signal measurement captured in response to a prompt of a second prompt type can be compared in order to generate a mental health prediction of the user. For example, EEG signal measurements corresponding to different respective prompt types can be compared to diagnose one or more mental illnesses, e.g., major depressive disorder, bipolar disorder, or anxiety.

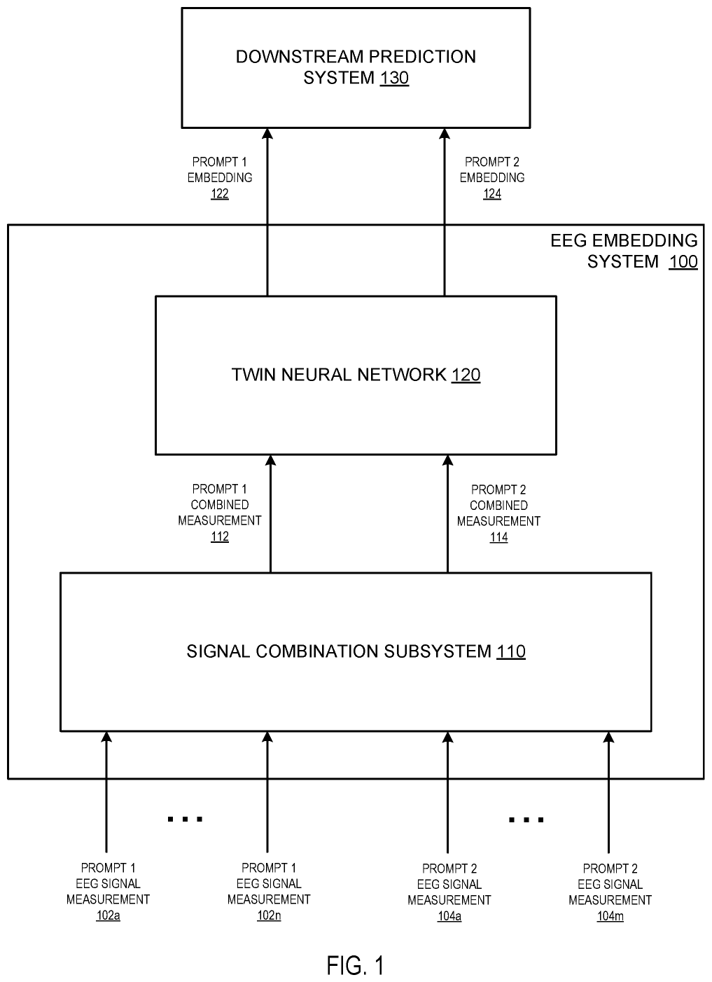

FIG. 1 is a diagram of an example EEG embedding system 100. The EEG embedding system 100 is an example of a system implemented as computer programs on one or more computers in one or more locations, in which the systems, components, and techniques described below can be implemented. In this specification, an embedding is an ordered collection of numeric values that represents an input in a particular embedding space. For example, an embedding can be a vector of floating point or other numeric values that has a fixed dimensionality.

The EEG embedding system 100 receives as input multiple EEG signal measurements, corresponding to respective different EEG trials, for each prompt type of an

EEG task. In particular, for an EEG task that has two prompt types, the EEG embedding system 100 receives as input multiple EEG signal measurements 102a-n corresponding to a first prompt type of the two prompt types and multiple EEG signal measurements 104a-m corresponding to a second prompt type of the two prompt types. In this example, there are N EEG signal measurements corresponding to the first prompt type and MEEG signal measurements corresponding to the second prompt type, where N>1 and M>1.

The EEG embedding system 100 generates a respective embedding 122 and 124 of the EEG signal measurements corresponding to the two different prompt types. Note that, while the example illustrated in FIG. 1 has two prompt types, in general an EEG task can have more prompt types, e.g., 3, 5, 10, or 50.

The EEG embedding system 100 includes a signal combination subsystem 110 and a twin neural network 120.

The signal combination subsystem 110 receives the EEG signal measurements 102a-n and the EEG signal measurements 104a-m, combines the N EEG signal measurements 102a-n corresponding to the first prompt type to generate a combined measurement 112 corresponding to the first prompt type, and combines the MEEG signal measurements 104a-m corresponding to the second prompt type to generate a combined measurement 114 corresponding to the second prompt type.

An EEG signal measurement corresponding to a single EEG trial can have a high degree of noise. For instance, a single EEG signal measurement might not be an accurate representation of the typical response of the brain of the user to the corresponding prompt type of an EEG task. Therefore, in order to generate an embedding 122 or 124 that represents the response of the brain of the user to prompts of a particular prompt type, the signal EEG embedding system 100 combines multiple different EEG signal measurement corresponding to the particular prompt type that were gathered during respective different EEG trials. Because the combined measurement is a combination of multiple different signal measurements gathered during independent EEG trials, the combined measurement does not have as much noise as a single EEG signal measurement. For example, the combined measurement is more representative of the response of the brain of the user to the particular prompt type.

In some implementations, the signal combination subsystem 110 determines an average of the EEG signal measurements corresponding to a particular prompt type; that is, the combined measurement 112 corresponding to the first prompt type is a time series that represents the average of the N time series EEG signal measurements, and the combined measurement 114 corresponding to the second prompt type is a time series that represents the average of the M time series EEG signal measurements 104a-m. In some other implementations, the signal combination subsystem 110 processes each EEG signal measurement corresponding to a particular prompt type using a neural network, e.g., a transformer neural network, to generate a single fixed-length embedding of the EEG signal measurements. That is, the combined measurements 112 and 114 can each be a fixed-length embedding in a learned embedding space.

The signal combination subsystem 110 provides the combined measurements 112 and 114 to the twin neural network 120, which processes the combined measurements 112 and 114 to generate the respective embeddings 122 and 124 of the EEG signal measurements corresponding to the two prompt types.

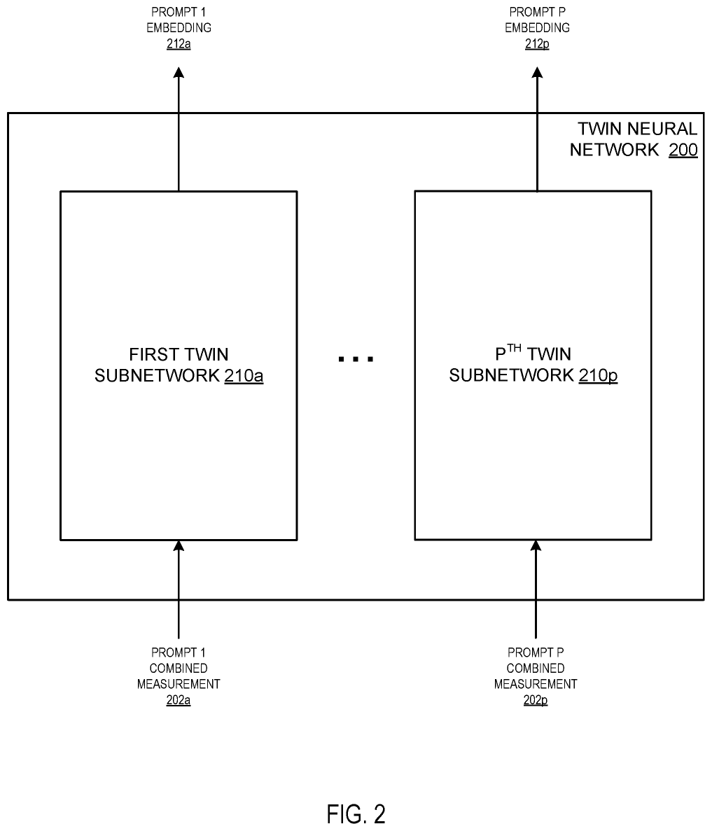

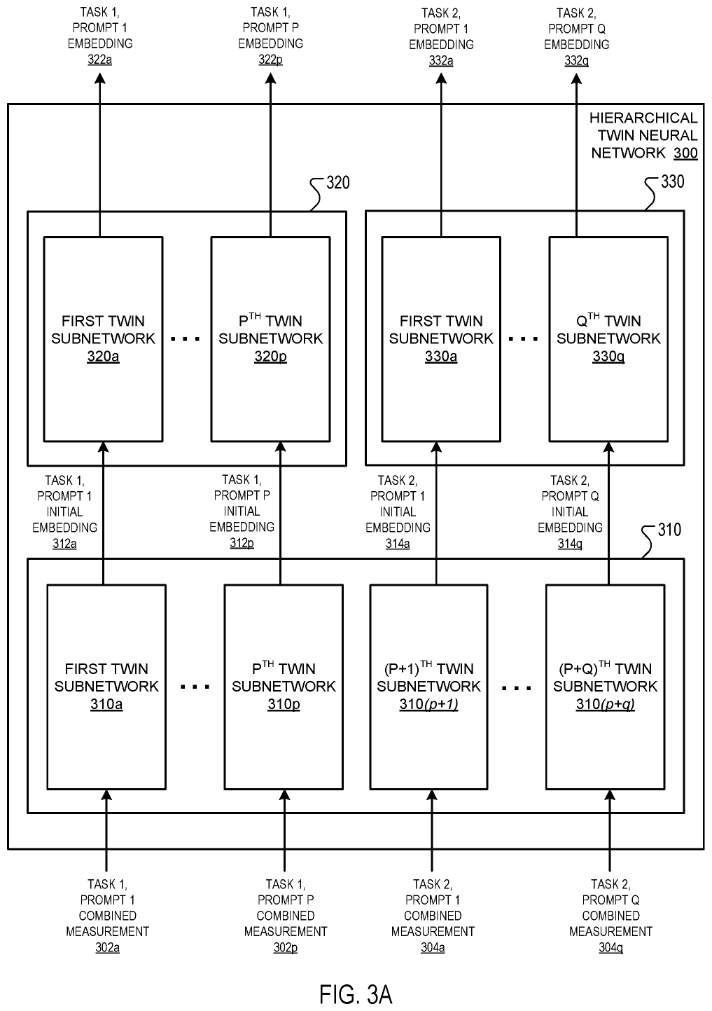

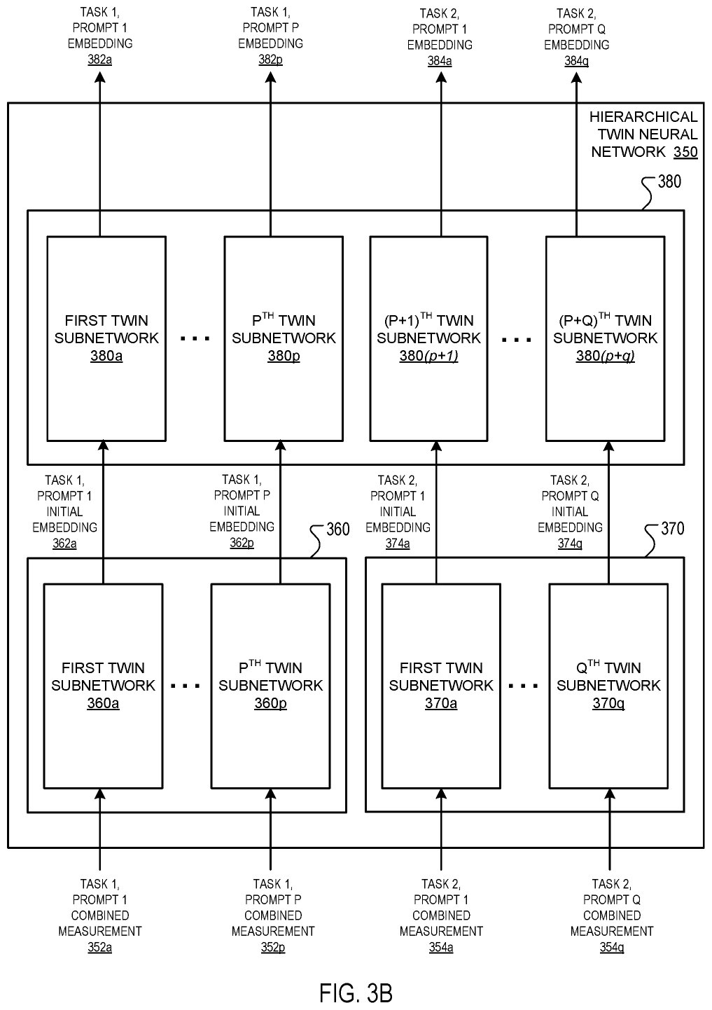

As used in this specification, the term “twin neural network” refers to a neural network that has multiple different subnetworks that have the same network architecture and the same parameter values, and that process different inputs. For example, the twin neural network 120 can have two or more different subnetworks that have the same network architecture and the same parameter values, where a first subnetwork processes the combined measurement 112 to generate the embedding 122 corresponding to the first prompt type and a second subnetwork processes the combined measurement 114 to generate the embedding 124 corresponding to the second prompt type. Because each subnetwork has the same architecture and the same parameter values, each subnetwork can extract the same features from the respective combined signal measurements to generate an embedding corresponding to the respective prompt of the EEG task. Example twin neural networks are discussed in more detail below in reference to FIG. 2 and FIGS. 3A and 3B.

After generating the two embeddings 122 and 124, the EEG embedding system 100 can provide the embeddings 122 and 124 to a downstream prediction system 130 to generate a mental health prediction for the user. For example, the downstream prediction system 130 can be a machine learning model, e.g., a neural network, that processes the embeddings 122 and 124 to predict a likelihood that the user has a particular mental health disorder. The neural network can be trained to extract information that characterizes a dissimilarity between the respective embeddings of the prompt types of the EEG task.

In some implementations, the twin neural network 120 and the downstream prediction system 130 can be trained jointly. That is, a training system can process training example that include EEG signal measurements using the twin neural network 120 to generate embeddings of the EEG signal measurements, and then process the embeddings using the downstream prediction system 130 to generate a training prediction. The training system can then determine an error in the training prediction, and backpropagate the error through both the downstream prediction system 130 and the twin neural network 120 to determine updates to the parameters values of the twin neural network 120 and the downstream prediction system 130.

In some implementations, the EEG embedding system 100 can provide the same embeddings 122 and 124 to multiple different downstream prediction systems 130. For example, the information encoded in the embeddings 122 and 124 can be useful to generate a prediction for multiple different mental health disorders corresponding to respective downstream systems.

FIG. 2 is a diagram of an example twin neural network 200. The twin neural network 200 is an example of a system implemented as computer programs on one or more computers in one or more locations, in which the systems, components, and techniques described below can be implemented.

The twin neural network 200 is configured to obtain P combined measurements 202a-p corresponding to P different prompt types of an EEG task, and process the combined measurements 202a-p to generate P different embeddings 212a-p corresponding to the respective prompt types. Each combined measurement 202 characterizes one or more different EEG signal measurements collected by measuring the brain of a user during respective EEG trials, where each EEG signal measurement was obtained in response to providing the user a prompt of the respective prompt type. For example, the twin neural network 200 can be a component of an EEG embedding system, e.g., the EEG embedding system 100 depicted in FIG. 1.

The twin neural network 200 includes P different twin subnetworks 210a-p. Each twin subnetwork has the same network architecture and parameter values; that is, each twin subnetwork 210a-p has the same number of nodes, where the nodes within each twin subnetwork 210a-p have the same relationship to each other and each corresponding node in the twin subnetworks 210a-p has the same parameter value. Each twin subnetwork 210a-p is configured to process a respective combined measurement 202 corresponding to a particular prompt type of the EEG task in order to generate a respective embedding 212 corresponding to the particular prompt type. Thus, each combined measurement 202a-p is processed using the same network architecture, so that the same information can be extracted from the combined measurements 202a-p and encoded in the embeddings 212a-p.

In some implementations, the combined measurements 202a-p are each a one-dimensional time series that represents the one or more EEG signal measurements corresponding to the respective prompt type, e.g., an average of the one or more EEG signal measurements. In some such implementations, each twin subnetwork 210a-p can include one or more one-dimensional convolutional neural network layers. Each one-dimensional convolutional neural network layer can process the time series using a respective one-dimensional convolutional filter.

Each combined measurement 202a-p can be a one-dimensional time series that includes multiple channels, where each channel corresponds to a different EEG sensor placed on the scalp of the user. In some implementations, for each one-dimensional convolutional neural network layer of the twin subnetworks 210a-p, the layer processes each channel of the time series with the same one-dimensional convolutional filter. In some other implementations, each one-dimensional convolutional filter has multiple channels, where each channel of the filter has different values and corresponds to a different channel of the combined measurements 202a-p.

The twin neural network 200 can provide the embeddings 212a-p to a downstream system that processes the embeddings 212a-p to generate a mental health prediction for the user, e.g., the downstream prediction system 130 depicted in FIG. 1.

In some implementations, the twin neural network 200 also includes a combining subnetwork that obtains each embedding 212a-p and combines them to generate a single user embedding. The user embedding can encode information corresponding to each prompt type of the EEG task, extracted from the respective embedding 212. The twin neural network 200 can then provide the user embedding to a downstream system to generate a mental health prediction for the user.

In some implementations, a twin neural network can generate embeddings corresponding to prompt types of multiple different EEG tasks. That is, for each EEG task of multiple different EEG tasks, and for each prompt type of one or more prompt types corresponding to the EEG task, the twin neural network can obtain a combined measurement representing one or more EEG signal measurements captured in response to providing a prompt of the prompt type to the user and process the combined measurement to generate a respective embedding corresponding to the prompt type and EEG task.

In some such implementations, the twin neural network can be a “flat” neural network; that is, there is a single layer of twin subnetworks, similar to the twin neural network 200 depicted in FIG. 2, that each have the same network architecture and parameter values, and each twin subnetwork processes a combined measurement corresponding to a respective EEG task and a respective prompt type of the EEG task. In other words, there is a single twin subnetwork corresponding to each combined embedding, and each combined embedding is processed using the exact same network architecture and parameter values.

In some other such implementations, the twin neural network can be a “hierarchical” neural network; that is, there are multiple groups of twin subnetworks, where the twin subnetworks in the same group have the same network architecture and parameter values, while twin subnetworks in different groups have different network architectures and/or parameter values. Example hierarchical twin neural networks are described in more detail below with respect to FIGS. 3A and 3B.

FIG. 3A is a diagram of an example hierarchical twin neural network 300 for generating EEG embeddings corresponding to multiple EEG tasks. The hierarchical twin neural network 300 is an example of a system implemented as computer programs on one or more computers in one or more locations, in which the systems, components, and techniques described below can be implemented.

The hierarchical twin neural network 300 generates a respective embedding 322a-p and 332a-q of the combined measurements corresponding to two different EEG tasks, where the first EEG task has P different prompt types and the second EEG task has Q different prompt types, where P≥1 and Q≥1. Note that, while the example illustrated in FIG. 1 has two EEG tasks, in general a hierarchical twin neural network can process combined embeddings corresponding to any number of EEG tasks, e.g., 3, 5, 10, or 50.

The hierarchical twin neural network 300 receives as input combined measurements 302a-p and 304a-q corresponding to each prompt type of each EEG task. The hierarchical twin neural network 300 processes each combined measurement 302a-p and 304a-q using a respective twin subnetwork 310a-(p+q) in a first group of twin subnetworks 310 that each have the same network architecture and parameter values. That is, there are P+Q twin subnetworks 310 in the first group of twin subnetworks 310 that each has the same network architecture and parameter values. Each twin subnetwork 310 generates a respective initial embedding 312a-p and 314a-q corresponding to the respective EEG task and prompt type. The initial embeddings 312a-p correspond to the first EEG task and the initial embeddings 314a-q correspond to the second EEG task.

For each initial embedding 312a-p corresponding to the first EEG task, the hierarchical twin neural network 300 processes the initial embedding using a respective twin subnetwork 320a-p in a second group of twin subnetworks 320 that each have the same network architecture and parameter values as each other, but a different network architecture and/or parameter values than the twin subnetworks in the first group of twin subnetworks 310. Each twin subnetwork 320a-p in the second group of twin subnetworks 320 generates an embedding 322a-p corresponding to the first EEG task and a respective prompt type of the first EEG task. For each initial embedding 314a-q corresponding to the second EEG task, the hierarchical twin neural network 300 processes the initial embedding using a respective twin subnetwork 330a-q in a third group of twin subnetworks 330 that each have the same network architecture and parameter values as each other, but a different network architecture and/or parameter values than the twin subnetworks in the first group of twin subnetworks 310 and the second group of twin subnetworks 320. Each twin subnetwork 330a-q in the third group of twin subnetworks 330 generates an embedding 332a-q corresponding to the second EEG task and a respective prompt type of the second EEG task.

Thus, the hierarchical twin neural network 300 P+Q embeddings 322a-p and 332a-q, each corresponding to a respective prompt type of either the first EEG task or the second EEG task.

FIG. 3B is a diagram of another example hierarchical twin neural network 350 for generating EEG embeddings corresponding to multiple EEG tasks. The hierarchical twin neural network 350 is an example of a system implemented as computer programs on one or more computers in one or more locations, in which the systems, components, and techniques described below can be implemented.

The hierarchical twin neural network 350 generates a respective embedding 382a-p and 384a-q of the combined measurements corresponding to two different EEG tasks, where the first EEG task has P different prompt types and the second EEG task has Q different prompt types, where P≥1 and Q≥1.

The hierarchical twin neural network 350 receives as input combined measurements 352a-p corresponding to each prompt type of the first EEG task and combined measurements 354a-q corresponding to each prompt type of the second EEG task.

The hierarchical twin neural network 300 processes each combined measurement 352a-p corresponding to the first EEG task using a respective twin subnetwork 360a-p in a first group of twin subnetworks 360 that each have the same network architecture and parameter values. That is, the first group of twin subnetworks 360 includes P twin subnetworks each corresponding to a respective prompt type of the first EEG task. Each twin subnetwork 360a-p in the first group of twin subnetworks 360 generates a respective initial embedding 362a-p corresponding to the first EEG task and a respective prompt type of the first EEG task.

The hierarchical twin neural network 300 processes each combined measurement 354a-p corresponding to the second EEG task using a respective twin subnetwork 370a-q in a second group of twin subnetworks 370 that each have the same network architecture and parameter values as each other, but a different network architecture and/or parameter values than the twin subnetworks in the first group of twin subnetworks 360. That is, the second group of twin subnetworks 370 includes Q twin subnetworks each corresponding to a respective prompt type of the second EEG task. Each twin subnetwork 370a-q in the second group of twin subnetworks 370 generates a respective initial embedding 374a-q corresponding to the second EEG task and a respective prompt type of the second EEG task.

The hierarchical twin neural network 350 processes each initial embedding 352a-p and 374a-q corresponding to either task using a respective twin subnetwork 380a-(p+q) in a third group of twin subnetworks 380 that each have the same network architecture and parameter values as each other, but a different network architecture and/or parameter values than the twin subnetworks in the first group of twin subnetworks 360 and the second group of twin subnetworks 370. That is, there are P+Q twin subnetwork in the third group of twin subnetworks 380 that each generate an embedding 382a-p and 384a-q corresponding to a respective EEG task and a respective prompt type of the respective EEG task. The embeddings 382a-p correspond to prompt types of the first EEG task and the embeddings 384a-q correspond to prompt types of the second EEG task.

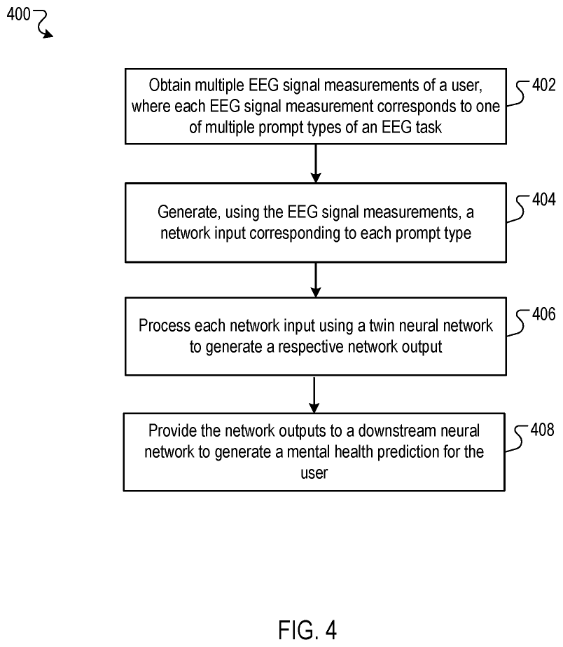

FIG. 4 is a flow diagram of an example process 400 for generating a mental health prediction for a user. For convenience, the process 400 will be described as being performed by a system of one or more computers located in one or more locations. For example, an EEG embedding system, e.g., the EEG embedding system 100 depicted in FIG. 1, appropriately programmed in accordance with this specification, can perform the process 400.

The system obtains multiple EEG signal measurements of a user, where each EEG signal measurement corresponds to one of multiple prompt types of an EEG task (step 402).

The system generates, using the EEG signal measurements, a network input corresponding to each prompt type of the EEG task (step 404). In some implementations, the system generates a network input corresponding to a particular prompt type of the EEG task by determining an average of the EEG signal measurements corresponding to the particular prompt type. In some other implementations, the system generates a network input corresponding to a particular prompt type of the EEG task by processing each EEG signal measurement corresponding to the particular prompt type using a neural network, e.g., a transformer neural network, to generate an embedding of the EEG signal measurements corresponding to the particular prompt type.

The system processes each network input using a twin neural network to generate a respective network output each corresponding to a different prompt type of the EEG task (step 406). The twin neural network include multiple subnetworks that each have a matching set of parameter values. In some implementations, e.g., when each network input includes a representative

EEG signal corresponding to the respective prompt type of the EEG task, the system can process each network input using one or more one-dimensional convolutional neural network layers. In some such implementations, the representative EEG signal can include multiple channels each corresponding to a different EEG sensor, and the system can process, for each of one or more convolutional neural network layers, each channel of the representative EEG signal using the same one-dimensional convolutional filter.

The system provides the network outputs to a downstream neural network to generate a mental health prediction for the user (step 408). In some implementations, the mental health prediction characterizes a likelihood that the user has a particular mental health disorder. In some implementations, the system can provide the network outputs to multiple different downstream neural networks that each process the network outputs to generate respective mental health predictions for the user, e.g., to generate a likelihood that the user has a respective different mental health disorder.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

In addition to the embodiments described above, the following embodiments are also innovative:

Embodiment 1 is a method comprising:

obtaining a plurality of electroencephalogram (EEG) signal measurements of a user, wherein each EEG signal measurement corresponds to one of a plurality of prompt types of an EEG task;

generating, from the plurality of EEG signal measurements, a plurality of network inputs each corresponding to a different prompt type of the plurality of prompt types of the EEG task;

processing the network inputs using a twin neural network to generate respective network outputs each corresponding to a different prompt type of the plurality of prompt types of the EEG task, the twin network processing each network input using a different subnetwork of a plurality of subnetworks of the twin neural network, wherein each of the plurality of subnetworks has a matching set of parameter values; and

providing the network outputs to a downstream neural network to generate a mental health prediction for the user.

Embodiment 2 is the method of embodiment 1, wherein generating a network input corresponding to a particular prompt type of the EEG task comprises determining an average of the EEG signal measurements corresponding to the particular prompt type.

Embodiment 3 is the method of any one of embodiments 1 or 2, wherein generating a network input corresponding to a particular prompt type of the EEG task comprises processing each EEG signal measurement corresponding to the particular prompt type using a transformer neural network to generate an embedding of the EEG signal measurements corresponding to the particular prompt type.

Embodiment 4 is the method of any one of embodiments 1-3, wherein:

each network input comprises a representative EEG signal corresponding to the respective prompt type of the EEG task; and

processing a network input using a subnetwork of the neural network comprises processing the representative EEG signal using one or more one-dimensional convolutional neural network layers.

Embodiment 5 is the method of embodiment 4, wherein:

the representative EEG signal comprises a plurality of channels each corresponding to a different EEG sensor; and

processing the representative EEG signal using a one-dimensional convolutional neural network layer comprises processing each channel of the representative EEG signal using a same one-dimensional convolutional filter.

Embodiment 6 is the method of any one of embodiments 1-5, wherein the mental health prediction characterizes a likelihood that the user has a particular mental health disorder.

Embodiment 7 is the method of embodiment 6, further comprising:

processing the network outputs using one or more second downstream neural networks to generate respective second mental health predictions for the user, wherein each second mental health prediction characterizes a likelihood that the user has a respective different mental health disorder.

Embodiment 8 is the method of any one of embodiment 1-7, wherein the network inputs are first network inputs, the network outputs are first network outputs, and the method further comprises:

obtaining a plurality of second EEG signal measurements of a user, wherein each second EEG signal measurement corresponds to one of a plurality of prompt types of a second EEG task; generating, from the plurality of second EEG signal measurements, a plurality of second network inputs each corresponding to a different prompt type of the plurality of prompt types of the second EEG task;

processing the second network inputs using the twin neural network to generate respective second network outputs each corresponding to a different prompt type of the plurality of prompt types of the second EEG task.

Embodiment 9 is the method of embodiment 8, wherein processing the first network inputs and the second network inputs using the twin neural network comprises:

processing, for each first network input and each second network input, the network input using a respective first subnetwork from a group of first subnetworks to generate a respective intermediate output, wherein each first subnetwork has a matching set of parameter values;

processing, for each intermediate output corresponding to a first network input, the intermediate output using a respective second subnetwork from a group of second subnetworks to generate a respective first network output, wherein each second subnetwork has a matching set of parameter values; and

processing, for each intermediate output corresponding to a second network input, the intermediate output using a respective third subnetwork from a group of third subnetworks to generate a respective second network output, wherein each third subnetwork has a matching set of parameter values.

Embodiment 10 is the method of any one of embodiments 8 or 9, wherein processing the first network inputs and the second network inputs using the twin neural network comprises:

processing, for each first network input, the first network input using a respective first subnetwork from a group of first subnetworks to generate a respective first intermediate output, wherein each first subnetwork has a matching set of parameter values;

processing, for each second network input, the second network input using a respective second subnetwork from a group of second subnetworks to generate a respective second intermediate output, wherein each second subnetwork has a matching set of parameter values; and

processing, for each first intermediate output and each second intermediate output, the intermediate output using a respective third subnetwork from a group of third subnetworks to generate a respective network output, wherein each third subnetwork has a matching set of parameter values.

Embodiment 11 is a system comprising: one or more computers and one or more storage devices storing instructions that are operable, when executed by the one or more computers, to cause the one or more computers to perform the method of any one of embodiments 1 to 10.

Embodiment 12 is a computer storage medium encoded with a computer program, the program comprising instructions that are operable, when executed by data processing apparatus, to cause the data processing apparatus to perform the method of any one of embodiments 1 to 10.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method comprising:
obtaining a plurality of electroencephalogram (EEG) signal measurements of a user, wherein each EEG signal measurement corresponds to one of a plurality of prompt types of an EEG task;
generating, from the plurality of EEG signal measurements, a plurality of network inputs each corresponding to a different prompt type of the plurality of prompt types of the EEG task;
processing the network inputs using a twin neural network to generate respective network outputs each corresponding to a different prompt type of the plurality of prompt types of the EEG task, the twin network processing each network input using a different subnetwork of a plurality of subnetworks of the twin neural network, wherein each of the plurality of subnetworks has a matching set of parameter values; and
providing the network outputs to a downstream neural network to generate a mental health prediction for the user.

2. The method of claim 1, wherein generating a network input corresponding to a particular prompt type of the EEG task comprises determining an average of the EEG signal measurements corresponding to the particular prompt type.

3. The method of claim 1, wherein generating a network input corresponding to a particular prompt type of the EEG task comprises processing each EEG signal measurement corresponding to the particular prompt type using a transformer neural network to generate an embedding of the EEG signal measurements corresponding to the particular prompt type.

4. The method of claim 1, wherein:
each network input comprises a representative EEG signal corresponding to the respective prompt type of the EEG task; and
processing a network input using a subnetwork of the neural network comprises processing the representative EEG signal using one or more one-dimensional convolutional neural network layers.

5. The method of claim 4, wherein:
the representative EEG signal comprises a plurality of channels each corresponding to a different EEG sensor; and
processing the representative EEG signal using a one-dimensional convolutional neural network layer comprises processing each channel of the representative EEG signal using a same one-dimensional convolutional filter.

6. The method of claim 1, wherein the mental health prediction characterizes a likelihood that the user has a particular mental health disorder.

7. The method of claim 6, further comprising:
processing the network outputs using one or more second downstream neural networks to generate respective second mental health predictions for the user, wherein each second mental health prediction characterizes a likelihood that the user has a respective different mental health disorder.

8. The method of claim 1, wherein the network inputs are first network inputs, the network outputs are first network outputs, and the method further comprises:
obtaining a plurality of second EEG signal measurements of a user, wherein each second EEG signal measurement corresponds to one of a plurality of prompt types of a second EEG task;
generating, from the plurality of second EEG signal measurements, a plurality of second network inputs each corresponding to a different prompt type of the plurality of prompt types of the second EEG task;
processing the second network inputs using the twin neural network to generate respective second network outputs each corresponding to a different prompt type of the plurality of prompt types of the second EEG task.

9. The method of claim 8, wherein processing the first network inputs and the second network inputs using the twin neural network comprises:
processing, for each first network input and each second network input, the network input using a respective first subnetwork from a group of first subnetworks to generate a respective intermediate output, wherein each first subnetwork has a matching set of parameter values;
processing, for each intermediate output corresponding to a first network input, the intermediate output using a respective second subnetwork from a group of second subnetworks to generate a respective first network output, wherein each second subnetwork has a matching set of parameter values; and
processing, for each intermediate output corresponding to a second network input, the intermediate output using a respective third subnetwork from a group of third subnetworks to generate a respective second network output, wherein each third subnetwork has a matching set of parameter values.

10. The method of claim 8, wherein processing the first network inputs and the second network inputs using the twin neural network comprises:
processing, for each first network input, the first network input using a respective first subnetwork from a group of first subnetworks to generate a respective first intermediate output, wherein each first subnetwork has a matching set of parameter values;
processing, for each second network input, the second network input using a respective second subnetwork from a group of second subnetworks to generate a respective second intermediate output, wherein each second subnetwork has a matching set of parameter values; and
processing, for each first intermediate output and each second intermediate output, the intermediate output using a respective third subnetwork from a group of third subnetworks to generate a respective network output, wherein each third subnetwork has a matching set of parameter values.

11. A system comprising one or more computers and one or more storage devices storing instructions that are operable, when executed by the one or more computers, to cause the one or more computers to perform a method comprising:
obtaining a plurality of electroencephalogram (EEG) signal measurements of a user, wherein each EEG signal measurement corresponds to one of a plurality of prompt types of an EEG task;
generating, from the plurality of EEG signal measurements, a plurality of network inputs each corresponding to a different prompt type of the plurality of prompt types of the EEG task;
processing the network inputs using a twin neural network to generate respective network outputs each corresponding to a different prompt type of the plurality of prompt types of the EEG task, the twin network processing each network input using a different subnetwork of a plurality of subnetworks of the twin neural network, wherein each of the plurality of subnetworks has a matching set of parameter values; and
providing the network outputs to a downstream neural network to generate a mental health prediction for the user.

12. The system of claim 11, wherein:
each network input comprises a representative EEG signal corresponding to the respective prompt type of the EEG task; and
processing a network input using a subnetwork of the neural network comprises processing the representative EEG signal using one or more one-dimensional convolutional neural network layers.

13. The system of claim 11, wherein the network inputs are first network inputs, the network outputs are first network outputs, and the method further comprises:
obtaining a plurality of second EEG signal measurements of a user, wherein each second EEG signal measurement corresponds to one of a plurality of prompt types of a second EEG task;
generating, from the plurality of second EEG signal measurements, a plurality of second network inputs each corresponding to a different prompt type of the plurality of prompt types of the second EEG task;
processing the second network inputs using the twin neural network to generate respective second network outputs each corresponding to a different prompt type of the plurality of prompt types of the second EEG task.

14. The system of claim 13, wherein processing the first network inputs and the second network inputs using the twin neural network comprises:
processing, for each first network input and each second network input, the network input using a respective first subnetwork from a group of first subnetworks to generate a respective intermediate output, wherein each first subnetwork has a matching set of parameter values;
processing, for each intermediate output corresponding to a first network input, the intermediate output using a respective second subnetwork from a group of second subnetworks to generate a respective first network output, wherein each second subnetwork has a matching set of parameter values; and
processing, for each intermediate output corresponding to a second network input, the intermediate output using a respective third subnetwork from a group of third subnetworks to generate a respective second network output, wherein each third subnetwork has a matching set of parameter values.

15. The system of claim 13, wherein processing the first network inputs and the second network inputs using the twin neural network comprises:
processing, for each first network input, the first network input using a respective first subnetwork from a group of first subnetworks to generate a respective first intermediate output, wherein each first subnetwork has a matching set of parameter values;
processing, for each second network input, the second network input using a respective second subnetwork from a group of second subnetworks to generate a respective second intermediate output, wherein each second subnetwork has a matching set of parameter values; and
processing, for each first intermediate output and each second intermediate output, the intermediate output using a respective third subnetwork from a group of third subnetworks to generate a respective network output, wherein each third subnetwork has a matching set of parameter values.

16. One or more non-transitory computer storage media encoded with computer program instructions that when executed by a plurality of computers cause the plurality of computers to perform operations comprising:
obtaining a plurality of electroencephalogram (EEG) signal measurements of a user, wherein each EEG signal measurement corresponds to one of a plurality of prompt types of an EEG task;
generating, from the plurality of EEG signal measurements, a plurality of network inputs each corresponding to a different prompt type of the plurality of prompt types of the EEG task;
processing the network inputs using a twin neural network to generate respective network outputs each corresponding to a different prompt type of the plurality of prompt types of the EEG task, the twin network processing each network input using a different subnetwork of a plurality of subnetworks of the twin neural network, wherein each of the plurality of subnetworks has a matching set of parameter values; and
providing the network outputs to a downstream neural network to generate a mental health prediction for the user.

17. The non-transitory computer storage media of claim 16, wherein:
each network input comprises a representative EEG signal corresponding to the respective prompt type of the EEG task; and
processing a network input using a subnetwork of the neural network comprises processing the representative EEG signal using one or more one-dimensional convolutional neural network layers.

18. The non-transitory computer storage media of claim 16, wherein the network inputs are first network inputs, the network outputs are first network outputs, and the operations further comprise:
obtaining a plurality of second EEG signal measurements of a user, wherein each second EEG signal measurement corresponds to one of a plurality of prompt types of a second EEG task;
generating, from the plurality of second EEG signal measurements, a plurality of second network inputs each corresponding to a different prompt type of the plurality of prompt types of the second EEG task;
processing the second network inputs using the twin neural network to generate respective second network outputs each corresponding to a different prompt type of the plurality of prompt types of the second EEG task.

19. The non-transitory computer storage media of claim 18, wherein processing the first network inputs and the second network inputs using the twin neural network comprises:
processing, for each first network input and each second network input, the network input using a respective first subnetwork from a group of first subnetworks to generate a respective intermediate output, wherein each first subnetwork has a matching set of parameter values;
processing, for each intermediate output corresponding to a first network input, the intermediate output using a respective second subnetwork from a group of second subnetworks to generate a respective first network output, wherein each second subnetwork has a matching set of parameter values; and
processing, for each intermediate output corresponding to a second network input, the intermediate output using a respective third subnetwork from a group of third subnetworks to generate a respective second network output, wherein each third subnetwork has a matching set of parameter values.

20. The non-transitory computer storage media of claim 18, wherein processing the first network inputs and the second network inputs using the twin neural network comprises:
processing, for each first network input, the first network input using a respective first subnetwork from a group of first subnetworks to generate a respective first intermediate output, wherein each first subnetwork has a matching set of parameter values;
processing, for each second network input, the second network input using a respective second subnetwork from a group of second subnetworks to generate a respective second intermediate output, wherein each second subnetwork has a matching set of parameter values; and
processing, for each first intermediate output and each second intermediate output, the intermediate output using a respective third subnetwork from a group of third subnetworks to generate a respective network output, wherein each third subnetwork has a matching set of parameter values.
